(19) United States
(12) Patent Application Publication
(10) Pub. No.: US 2008/0044842 A1
(43) Pub. Date: Feb. 21, 2008

(54) Biological Test Strip

(76) Inventors: Sung-Kwon Jung, Granger, IN (US); Steven C. Charlton, Osceola, IN (US)

Correspondence Address: NIXON PEABODY LLP, 161 N. CLARK STREET, 48TH FLOOR, CHICAGO, IL, 60601, US

(21) Appl. No.: 11/662,718

(22) PCT Filed: Sep. 19, 2005
(86) PCT No.: PCT/US05/33294

§ 371 Date: Mar. 16, 2007

Related U.S. Application Data
(60) Provisional application No. 60/611,470, filed on Sep. 20, 2004.

(51) Int. Cl. G01N 33/52; C12Q 1/00; C12Q 1/54; G01N 33/49

(52) U.S. Cl. 435/14; 156/60; 204/403.06; 422/68.1; 435/288.7; 435/4; 436/164

(57) ABSTRACT

A test sensor (10) for use in the determination of an analyte in a liquid sample. The test sensor (10) includes a base (14), a lid (12), and a test membrane (18) adhered to the base. A reagent (20) is contained within the test membrane (18). A mesh strip (22) is also included in the sensor and is adhered to the lid (12). An end of the mesh strip extends at least to the end of the lid, such that the mesh strip can move a liquid sample from the mesh strip to the reagent in the test membrane.

### FIELD OF THE INVENTION

The present invention relates generally to liquid sample monitoring devices and, more particularly, to the manufacture and design of a test sensor for use in determining the concentration of an analyte in a liquid sample.

### BACKGROUND OF THE INVENTION

Test sensors are often used in assays for determining the concentration of an analyte in a liquid sample. A liquid sample is deposited in a reaction area of the test sensor that includes a reagent. The sample and the reagent mix, producing a measurable reaction indicative of the concentration of the analyte in the liquid sample. The reaction is measured with a test device that receives the test sensor.

Testing for the concentration of glucose in blood is a common use for test sensors. Test sensors are also used for determining the concentration or presence of various other analytes (e.g., fructosamine, hemoglobin, cholesterol, glucose, alcohol, drugs, etc.) in a variety of body fluids (e.g., blood, interstitial fluid, saliva, urine, etc.). Test sensors including appropriate reagents can be used in the harvesting of most any liquid sample for the determination of the concentration of an analyte in that sample. Typically, these devices can employ either electrochemical testing or calorimetric testing. In an electrochemical assay, a regent is designed to react with glucose in the blood to create an oxidation current at electrodes disposed within the reaction area which is directly proportional to the concentration of glucose in the user's blood.

In a colorimeteric assay, the color change of a reaction area containing a reagent following contact with the sample is measured to determine the concentration of the analyte of interest in the sample. The degree of color change is measured using an optical sensor(s) that converts the degree of color change to electrical signals that are evaluated with diagnostic equipment. For example, the optical device may measure the amount of light reflected from, or transmitted through, the reaction area. In other embodiments of the present invention, the amount of infrared light absorbed by the reaction of the analyte in the sample and the reagent is measured. Colorimetric testing is described in detail in U.S. Pat. Nos. 6,181,417 (entitled “Photometric Readhead With Light Shaping Plate”), 5,518,689 (entitled “Diffuse Light Reflectance Readhead”) and 5,611,999 (entitled “Diffuse Light Reflectance Readhead”), each of which is incorporated herein by reference in its entirety.

One method of obtaining a blood sample and analyzing the sample is with a “top loading” sensor. In a top loading sensor, a drop of blood is obtained from the fingertip and then is loaded from above the sensor onto the reactive portion of the test sensor.

A drawback associated with the use of a “top loading” sensor is that the user may miss the reagent and the blood may drop onto a different part of the sensor, thereby wasting the sample. This requires the user to sometimes obtain numerous samples before obtaining an accurate reading.

Other methods of harvesting a blood sample with a test sensor involve using capillary action to draw the blood into the test sensor. A drawback of the capillary-type sensor is that compartments such as the side walls and the hole containing the reagent must be aligned in order to properly draw the sample into the capillary channel. It is time-consuming to perform this alignment and, thus, increases the manufacturing cost of the device.

### SUMMARY OF THE INVENTION

The present invention is a test sensor for use in the determination of an analyte in a liquid sample. The test sensor includes a base, a lid, and a test membrane adhered to the base. The test membrane contains a reagent. There is a mesh strip adhered to the lid which has an end extending at least as far as the end of the lid. The mesh strip is adapted to move a liquid sample from the mesh strip to the reagent in the test membrane.

The above summary of the present invention is not intended to represent each embodiment, or every aspect, of the present invention. This is the purpose of the figures and the detailed description which follow.

### BRIEF DESCRIPTION OF THE DRAWINGS

The foregoing and other advantages of the invention will become apparent upon reading the following detailed description and upon reference to the drawings.

FIG. 1 is a side view of a test sensor according to one embodiment of the present invention.

FIG. 2a is a top view of the embodiment of FIG. 1.

FIG. 2b is a top view of another embodiment of the present invention.

FIG. 3 is an exploded view of the embodiment of FIG. 1.

FIG. 4 is side view of the test sensor of FIG. 1 in combination with an optical sensor.

FIG. 5 is a flow chart describing the operation of an embodiment of the present invention.

FIG. 6 is a flow chart describing the operation of one embodiment of the present invention.

While the invention is susceptible to various modifications and alternative forms, specific embodiments are shown by way of example in the drawings and are described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DESCRIPTION OF ILLUSTRATIVE EMBODIMENTS

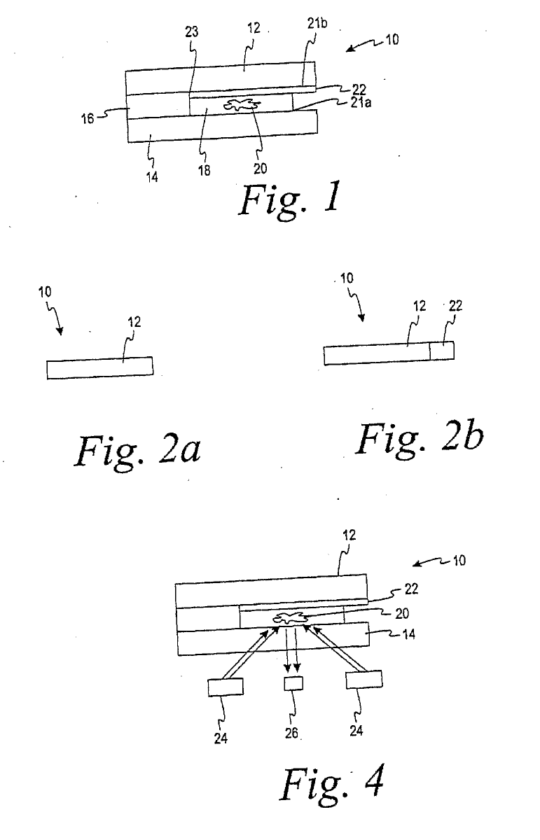

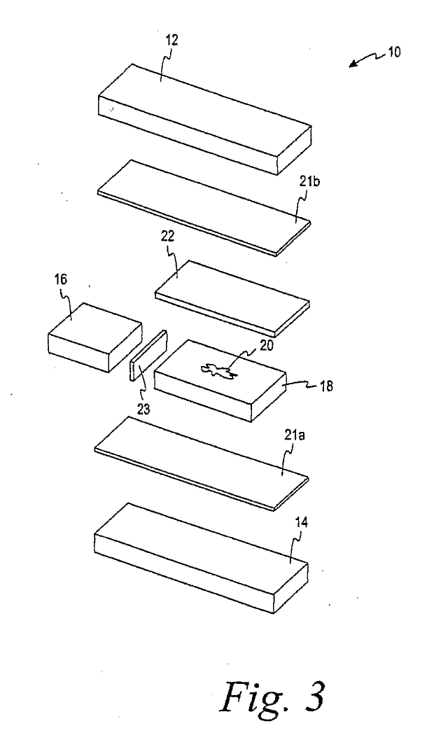

Turning now to the drawings, and initially to FIGS. 1-3, a test sensor 10 according to one embodiment of the invention is illustrated. The test sensor 10 is used in the harvesting and analysis of a liquid sample for determining the presence or concentration of an analyte in the liquid sample. In this embodiment, the test sensor 10 is an optical test sensor. The test sensor 10 includes a lid 12 and a base 14. The lid 12 and the base 14 are separated by a spacer 16. The spacer 16 is joined to the lid 12 and the base 14 by an adhesive. In optical testing, at least one of the lid 12 and the base 14 is substantially optically clear. The other of the lid 12 and the base 14 may be either substantially optically clear or opaque.

Attached to the base 14 is a test membrane 18 including a coated or impregnated reagent 20. The test membrane 18 is attached to the base 14 via a first adhesive layer 21a. Attached to the lid 12 via a second adhesive layer 21b is a mesh strip 22. The mesh strip 22 extends out at least as far as the edge of the lid 12. In some embodiments, as shown in FIG. 2b, the mesh strip 22b extends past the edge of the lid 12. The mesh strip 22 is adjacent to the test membrane 18, but the mesh strip 22 and the test membrane 18 are not attached to each other. Because the thickness of the spacer 16 is approximately the same as the combined thicknesses of the mesh strip 22 and the test membrane 18, the mesh strip 22 and the test membrane 18 are held in close proximity to each other. In some embodiments, the thickness of the spacer 16 is less than the combined thicknesses of the mesh strip 22 and the test membrane 18, causing the mesh strip 22 and the test membrane 18 to be pressed together. Also, in some embodiments, the ends of the test membrane 18 and the mesh strip 22 may be adhered to the spacer via a third adhesive layer 23. Also, in some embodiments, the adhesive used may be substantially optically clear.

Turning now to FIG. 4, the test sensor 10 will be shown in combination with a light source 24 and a read head 26 and one method for determining the amount of glucose in the blood sample will be described. In the embodiment shown in FIG. 4, after the blood is harvested and drawn into the test membrane 28, the light source 24 (of which two are shown) direct light through the optically clear base 14. The light transmits through the optically clear base 14 and the optically clear first layer of adhesive 21a, hits the reflective test membrane 18, and bounces back to the read head 26. The read head 26 then analyzes the light that has bounced off the test membrane 18 and a result is given to the user.

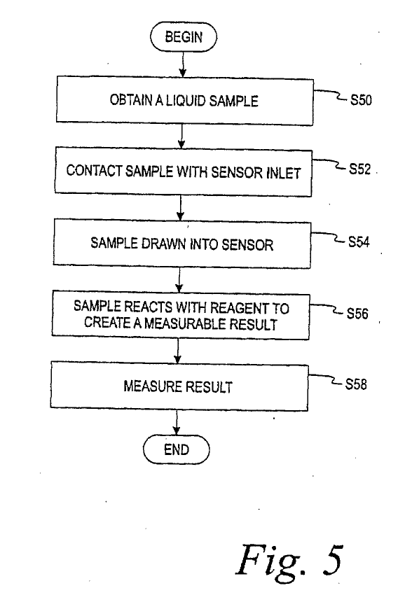

Turning now to FIG. 5, the operation of one embodiment of the present invention will be described. At step S50, the user obtains a liquid sample, in this case, blood from a finger. The test sensor 10 is then placed adjacent to the source of blood, with the mesh strip 22 touching the drop of blood at step S52. Via capillary action, the mesh strip 22 draws the blood into the test sensor 10 and deposits the blood onto the test membrane 18 at step S54. The blood mixes with the reagent 20 in the test membrane 18 to create a measurable result of the amount of an analyte, in this case, glucose in the blood (step S56). The result is then measured at step S58 by either using colorimetric or electrochemical analysis as described above.

The test sensor 10 described above has many advantages over the prior art. Unlike in “top loading” sensors, there is no need to drop the blood directly onto the test membrane. This is because the mesh strip 22 provides the capillaries needed to draw the blood into the test sensor 10, the need for dropping the blood directly onto the test membrane is obviated. Also, unlike other capillary-type test sensors, there is no need for the alignment of the various compartments. This is due to the fact that in the present test sensor 10, side walls are not needed for the capillary action, therefore, there is no need for alignment during the manufacturing process, which is time consuming and costly.

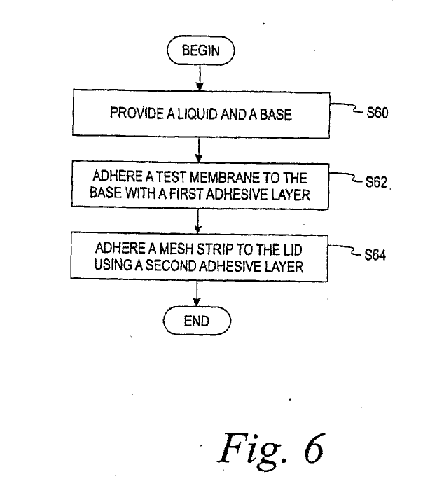

Turning now to FIG. 6, the manufacturing process of the test sensor 10 will be described. In step s60, the lid 12 and the base 14 are provided. The test membrane 18 is then adhered to the base 14 using the first adhesive layer 21a (step s62). The mesh strip 22 is then adhered to the lid 12 using the second adhesive layer 21b (step s64) such that the mesh strip 22 extends out at least as far as the end of the lid 12.

Although the test sensor 10 has been described as being an optical sensor, it should be understood that the test sensor 10 could also be an electrochemical sensor. If the test sensor 10 is an electrochemical sensor, none of the lid 12, the base 14, nor either the first or second adhesive layers 21a, 21b need to be optically clear. Also, the test membrane 18 does not need to be made of a reflective material. In an electrochemical test sensor, electrodes would extend into the test membrane 18, as is commonly known in the art.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the claimed invention, which is set forth in the following claims.

### Alternative Embodiment A

A test sensor for use in the determination of an analyte in a liquid sample, the test sensor comprising:
a base;
a lid;
a test membrane adhered to the base, the test membrane containing a reagent; and
a mesh strip adhered to the lid and having an end extending at least to the end of the lid, the mesh strip adapted to move a liquid sample from the mesh strip to the reagent in the test membrane.

### Alternative Embodiment B

The test sensor of embodiment A wherein the reagent is adapted to produce a calorimetric reaction indicative of the concentration of the analyte in the sample.

### Alternative Embodiment C

The test sensor of embodiment A wherein the reagent is adapted to produce an electrochemical reaction.

### Alternative Embodiment D

The test sensor of embodiment A wherein the analyte is glucose.

### Alternative Embodiment E

The test sensor of embodiment D adapted to measure the concentration of glucose in blood.

### Alternative Embodiment F

The test sensor of embodiment A wherein the at least one of the lid and the base is constructed of a substantially optically clear material.

### Alternative Embodiment G

The test sensor of embodiment A wherein the test membrane is adhered to the base with a substantially optically clear adhesive.

### Alternative Embodiment H

The test sensor of embodiment A further comprising a spacer adhered between the lid and the base.

### Alternative Embodiment I

The test sensor of embodiment I wherein the thickness of the spacer is approximately equal to the combined thickness of the test membrane and the mesh strip.

### Alternative Embodiment J

The test sensor of embodiment I wherein an end of the spacer is adhered to ends of the test membrane and the mesh strip.

### Alternative Embodiment K

The test sensor of embodiment A wherein the end of the mesh strip extends past the end of the lid.

### Alternative Embodiment L

A method for manufacturing a test sensor for use in the determination of an analyte in a liquid sample, the method comprising:
providing a lid and a base;
adhering a test membrane to the surface of the base, the test membrane including a reagent; and
adhering a mesh strip to a lid so that the mesh strip is adjacent to the test membrane and that an end of the mesh strip extends to at least an end of the lid.

### Alternative Embodiment M

The method of embodiment L wherein the mesh strip is adapted to use capillary action to draw the analyte into the test sensor, placing the analyte in contact with the reagent in the test membrane.

### Alternative Embodiment N

The method of embodiment L wherein the reagent is adapted to produce a colorimetric reaction indicative of the concentration of the analyte in the sample.

### Alternative Embodiment O

The method of embodiment L wherein the reagent is adapted to produce an electrochemical reaction.

### Alternative Embodiment P

The method of embodiment L wherein the analyte is glucose.

### Alternative Embodiment Q

The method of embodiment P adapted to measure the concentration of glucose in blood.

### Alternative Embodiment R

The method of embodiment L wherein the at least one of the lid and the base is constructed of a substantially optically clear material.

### Alternative Embodiment S

The method of embodiment L further comprising adhering the test membrane to the base with a substantially optically clear adhesive.

### Alternative Embodiment T

The method of embodiment L further comprising adhering a spacer between the lid and the base.

### Alternative Embodiment U

The method of embodiment T wherein the thickness of the spacer is approximately equal to the combined thickness of the test membrane and the mesh strip.

### Alternative Embodiment V

The method of embodiment U further comprising adhering an end of the spacer to ends of the test membrane and the mesh strip.

### Alternative Embodiment W

The method of embodiment L wherein the step of adhering a mesh strip to a lid comprises adhering the mesh strip such that an end of the mesh strip extends to at least an end of the lid.

### Alternative Embodiment X

A method for determining a concentration of an analyte in a liquid sample with a test sensor, the test sensor comprising a lid, a base, a test membrane adhered to the base, and a mesh strip adhered to the lid and extending at least to an end of the lid, the method comprising:
drawing the liquid sample into the test membrane with the mesh strip via capillary action; and
filling the test membrane with the liquid sample.

1. A test sensor for use in the determination of an analyte in a liquid sample, the test sensor comprising:
a base;
a lid;
a test membrane adhered to the base, the test membrane containing a reagent; and
a mesh strip adhered to the lid and having an end extending at least to the end of the lid, the mesh strip adapted to move a liquid sample from the mesh strip to the reagent in the test membrane.

2. The test sensor of claim 1, wherein the reagent is adapted to produce a calorimetric reaction indicative of the concentration of the analyte in the sample.

3. The test sensor of claim 1, wherein the reagent is adapted to produce an electrochemical reaction.

4. The test sensor of claim 1, wherein the analyte is glucose.

5. The test sensor of claim 4, adapted to measure the concentration of glucose in blood.

6. The test sensor of claim 1, wherein the at least one of the lid and the base is constructed of a substantially optically clear material.

7. The test sensor of claim 1, wherein the test membrane is adhered to the base with a substantially optically clear adhesive.

8. The test sensor of claim 1, further comprising a spacer adhered between the lid and the base.

9. The test sensor of claim 9, wherein the thickness of the spacer is approximately equal to the combined thickness of the test membrane and the mesh strip.

10. The test sensor of claim 9, wherein an end of the spacer is adhered to ends of the test membrane and the mesh strip.

11. The test sensor of claim 1, wherein the end of the mesh strip extends past the end of the lid.

12. A method for manufacturing a test sensor for use in the determination of an analyte in a liquid sample, the method comprising:
providing a lid and a base;
adhering a test membrane to the surface of the base, the test membrane including a reagent; and
adhering a mesh strip to a lid so that the mesh strip is adjacent to the test membrane and that an end of the mesh strip extends to at least an end of the lid.

13. The method of claim 12, wherein the mesh strip is adapted to use capillary action to draw the analyte into the test sensor, placing the analyte in contact with the reagent in the test membrane.

14. The method of claim 12, wherein the reagent is adapted to produce a colorimetric reaction indicative of the concentration of the analyte in the sample.

15. The method of claim 12, wherein the reagent is adapted to produce an electrochemical reaction.

16. The method of claim 12, wherein the analyte is glucose.

17. The method of claim 16, adapted to measure the concentration of glucose in blood.

18. The method of claim 12, wherein the at least one of the lid and the base is constructed of a substantially optically clear material.

19. The method of claim 12, further comprising adhering the test membrane to the base with a substantially optically clear adhesive.

20. The method of claim 12, further comprising adhering a spacer between the lid and the base.

21. The method of claim 20, wherein the thickness of the spacer is approximately equal to the combined thickness of the test membrane and the mesh strip.

22. The method of claim 21, further comprising adhering an end of the spacer to ends of the test membrane and the mesh strip.

23. The method of claim 12, wherein the step of adhering a mesh strip to a lid comprises adhering the mesh strip such that an end of the mesh strip extends to at least an end of the lid.

24. A method for determining a concentration of an analyte in a liquid sample with a test sensor, the test sensor comprising a lid, a base, a test membrane adhered to the base, and a mesh strip adhered to the lid and extending at least to an end of the lid, the method comprising:
drawing the liquid sample into the test membrane with the mesh strip via capillary action; and
filling the test membrane with the liquid sample.
